(19) United States
(12) Patent Application Publication
(10) Pub. No.: US 2006/0275278 A1
(43) Pub. Date: Dec. 7, 2006

(54) Method and ophthalmic formulation for eye protection or treatment

(76) Inventors: Camus Kar Man Choy, Hong Kong (CN); Hie Hua Wong, Hong Kong (CN); Iris Frances Forster Benzie, Hong Kong (CN); Emily Pik Yin Choy, Hong Kong (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/478,261

(22) Filed: Jun. 28, 2006

Related U.S. Application Data
(63) Continuation-in-part of application No. 11/142,396, filed on Jun. 2, 2005.

(51) Int. Cl. A61K 38/44; A61K 38/40; A61K 38/38; A61K 38/05; A61K 31/375; A61K 31/198; A61K 31/513

(52) U.S. Cl. 424/94.4; 514/6; 514/2; 514/18; 514/269; 514/561; 514/474; 514/567

(57) ABSTRACT

An ophthalmic composition for artificial tears useful for treating dry eye symptoms or for general eye protection. The composition includes a physiologically balanced salt solution and one or more anti-oxidative ingredients which collectively confer on said ophthalmic composition a total antioxidant capacity as a FRAP value of greater than 100 μmol/l, preferably greater than 150 μmol/l. The composition also has peak UV absorption in the range of 200-350 nm. Suitable anti-oxidative ingredient includes ascorbate (ascorbic acid), urate (uric acid), cysteine, glutathione, tyrosine, lactoferrin, transferrin, albumin, caeruloplasmin and lacrimal gland peroxidase.

### CROSS REFERENCE TO RELATED APPLICATION

This application is a continuation-in-part of U.S. application Ser. No. 11/142,396, filed Jun. 2, 2005, the entire contents of which are hereby incorporated by reference.

### FIELD OF THE INVENTION

The present invention relates to an ophthalmic composition useful in prevention and treatment of dry eye symptom or in improvement of human ocular health. More particularly, the invention relates to a composition for ophthalmic solutions, e.g. artificial tears, eyewash, and contact lens solution, which provides anti-oxidation capacity and UV absorption characteristics similar to those normally characterizing natural human tears and to methods of treating or preventing symptoms associated with dry eye syndrome or for improving ocular health in general.

### BACKGROUND OF THE INVENTION

Dry eye syndrome is one of the most common age-related ocular diseases. Over 50 million dry eye sufferers were found in USA and more than 30% of Chinese over the age of 65 will experience dry eye condition (Kirchner 1997; Lin et al. 2003). The prevalence of dry eye will further increase following the general aging of the population (Schein et al. 1997). Dry eye syndrome is related to decreased tear secretion, changed tear composition, or to an abnormality of the ocular surface leading to decreased tear stability (Lemp 1995).

Dry eye is believed to be due in part to the decrease in tear antioxidant protection (Augustin et al. 1995; Rieger 2001). Previous reports suggested that decreased tear antioxidant protection and increased reactive oxygen species (ROS)-induced ocular surface cell damage may be associated with dry eye problem. UV light is a powerful ROS generator and it can initiate processes of photo-oxidation and this is relevant to ocular health. Furthermore, contact lens wear may cause dry eye (Halliwell and Gutteridge 1999).

In dry eye syndrome, the patient experiences ocular discomfort, with burning, itchy, sandy or gritty sensations (Lemp 1995). If the problem is untreated, dry eye syndrome can lead to scarring or ulceration of the cornea, and possible loss of vision (Goto et al. 2002). Therefore, treating dry eye problem is important not only for improving comfort but also for improving the health of the cornea and maintenance of vision.

Regular use of artificial tears is a common treatment to relieve dry eye symptoms (Lemp 1987; Tsubota et al. 1996). Most artificial tear products, however, alleviate ocular discomfort temporarily, and do not arrest or reverse damage to the eye (Tsubota et al. 1996; Albietz 2001). Research on artificial tears mostly focused on how to prolong the duration of the lubrication effect of the artificial tears after application to the eye (Albietz 2001). Little research has been conducted to determine how the composition of artificial tears may be made to be like that of natural tears. To applicants' knowledge, presently none of the commercially available artificial tear products or other ophthalmic solutions emulate sufficiently some characteristics of natural tears, particularly, the anti-oxidative capacity and UV-filtering capacity.

### SUMMARY OF THE INVENTION

One object of the present invention, accordingly, is to provide a novel ophthalmic composition which provides anti-oxidative protection and ultraviolet radiation absorption characteristics similar to those existing in natural human tears. The composition comprises one or more anti-oxidative components in a sufficient amount so that the composition's anti-oxidative capacity is similar to that of natural tears. Preferably, the composition further comprises a UV-radiation absorbing component with a UV-absorption profile similar to natural tears. The UV-radiation absorbing component (or UV filter) and the anti-oxidative components (or antioxidant) may be the same substance or different substances.

In one embodiment, there is provided by the present invention an ophthalmic composition comprising a physiologically balanced salt solution and one or more anti-oxidative ingredients which collectively confer on said ophthalmic composition a total antioxidant capacity as the FRAP value greater than 100 μmol/l (preferably, greater than 150 μmol/l), where at least one ingredient has peak UV absorption in the range of 200-350 nm. Suitable anti-oxidative ingredient includes, but is not limited to, ascorbate (ascorbic acid), urate (uric acid), cysteine, glutathione, tyrosine, lactoferrin, transferrin, albumin, caeruloplasmin and lacrimal gland peroxidase. Of course, a single ingredient may function both as antioxidant and as UV filter. The process of making an ophthalmic fluid for clinical use based on the foregoing formulation of the invention is generally known to people skilled in the art, for example, in the pharmaceutical industry.

In another embodiment of the present invention, the ophthalmic composition comprises a physiologically balanced salt solution and at least one anti-oxidative ingredient selected from the group consisting of: ascorbate (ascorbic acid): about 10-80 μmol/l (preferably, 25 μmol/l); urate (uric acid): about 50-200 μmol/l (preferably, 100 μmol/l); cysteine: about 0.4-1.0 μmol/l (preferably, 0.7 μmol/l); glutathione: about 1.5-11.0 μmol/l (preferably, 4.0 μmol/l); tyrosine: about 1.0-6.0 μmol/l (preferably, 3.0 μmol/l); lactoferrin: about 1500-1790 μg/ml (preferably, 1645 μg/ml); transferrin: about 13-18 μg/ml (preferably, 15 μg/ml); albumin: about 37-47 μg/ml (preferably, 42 μg/ml); caeruloplasmin: about 8-14 μg/ml (preferably, 210 μg/ml); lacrimal gland peroxidase: about 4-7 μg/ml (preferably, 6 μg/ml). It is contemplated that the ophthalmic composition of the present invention may omit one or more of the foregoing listed ingredients and still achieves satisfactory results.

In another embodiment of the present invention, the ophthalmic composition further contains a viscosity-increasing component, which could be, but is not restricted to, from the group consisting of a cellulose ester, polyvinyl alcohol, providone, polyacrylic acid, hyaluronic acid, and chondroitin sulfate.

As another aspect of the present invention, there is provided a method for treating dry eye or improving ocular health in general, comprising a step of administering into the eye an ophthalmic composition comprising a physiologically balanced salt solution and one or more anti-oxidative ingredients which collectively confer on said ophthalmic composition a total antioxidant capacity as the FRAP value of greater than 100 μmol/l (preferably, greater than 150 μmol/l), where at least one ingredient has peak UV absorption in the range of 200-350 nm. The method may also comprise a step of mixing the physiologically balanced salt solution and the one or more anti-oxidative ingredients before the step of administering to the eye.

As another aspect of the present invention, there is provided a commercial artificial tear package, comprising an ophthalmic composition described above, containing a physiologically balanced salt solution and at least one anti-oxidative ingredient, wherein said one anti-oxidative ingredient is stored in a dry state in a first chamber and said physiologically balanced salt solution is stored in a second chamber.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### BRIEF DESCRIPTION OF DRAWINGS

FIGS. 1A-1B show the relationship between the total antioxidant capacity as the FRAP value and the tear flow rate in (A) younger and (B) older age subjects. These unpublished data indicate that the older group has lower tear flow rate than the younger group, but the antioxidant characteristics in both group is similar. The age-related decline on tear flow rate in older people leads to the dry eye development, and our data indicate that the use of artificial tears for dry eye treatment should not only provide water for eye lubrication, but also provide antioxidant(s) to maintain the antioxidant balance at the ocular surface.

FIGS. 2A-D show the UV absorbing profile of some commercially available ophthalmic solutions (A) Bion tears®, (B) Tear Naturale™ Free, (C) Blink™ eye drops, and pooled fresh human tears (D). These unpublished result show that none of the commercially available ophthalmic solutions tested have UV absorption characteristics similar to that of normal human tears.

### DETAILED DESCRIPTION OF THE INVENTION

I. Antioxidants in Human Tears

Natural Human Tear Preparation

Experiments were performed to determine the water-soluble antioxidant components in reflex tear collected from healthy young adults by capillary tubes and by Shirmer strips (without use of local anesthetic) and in basal tears collected by Shirmer strips (with use of local anesthetic), according to the procedure set forth in Choy et al. 2001. Briefly, twelve Chinese subjects (seven men, five women) aged from 22 to 29 years were recruited with their informed consent. All subjects had apparently normal general and ocular health. None were smokers or users of vitamin supplements, and none wore contact lenses. Subjects in this self-controlled study attended the clinic on one occasion only between the hours of 9 AM and 3 PM. Reflex tears were collected simultaneously from both eyes of each subject (one by capillary tube and the other by Shirmer strip). Basal tears were collected from the eye previously used for reflex tear collection by capillary tube 30 minutes after reflex tear collection.

After tear collection, the wet Schirmer strip was immediately put into the same tube as before, the tube was sealed to avoid evaporation, and the strip was reweighted to determine the weight of tears collection. This was translated into volume by using the tear density, which was calculated by weighing a known volume of the reflex tears collected by capillary tube from the same subject. Tears were eluted from strips using 50 μl phosphate buffer (HPLC mobile phase), and approximately 30 μl eluate was transferred into the sample cup for measurement. Approximately 30 μl of reflex tears collected by capillary tube were transferred directly into a sample cup.

Antioxidant Concentration Measurement

The sample cups were immediately placed in the autosampler compartment of an HPLC system (Millennium; Water Alliance, Milford, Mass.), and the antioxidants of interest (cysteine, ascorbate (ascorbic acid), glutathione, urate (uric acid), and tyrosine) were measured concurrently, according to the HPLC method of Gogia et al. (1998).

To check whether there was any antioxidant or contamination of the tear sample due to contact with the capillary tubes, Schirmer strips, or local anesthetic, three blank samples were prepared by collecting mobile phase into a capillary tube, absorbing mobile phase onto a Schirmer strip, eluting as described earlier, and adding 10 μl of local anesthetic to 55 μl mobile phase. In addition, recovery of added antioxidants was determined by measuring freshly prepared combined standard (containing all five antioxidants of interest), according to the same procedures used in preparation of the three blank samples. Combined standard (100 μM) was prepared freshly on each testing occasion by mixing equal parts of 500 μM standards of each antioxidant of interest (cysteine, ascorbate (ascorbic acid), glutathione, urate (uric acid), and tyrosine). The 500 μM standards were prepared fresh from stock solutions as follows: cysteine (200 mM) was prepared from solid (BDH Chemicals Led., Poole, UK) in extra pure water, and dissolving was facilitated by the addition of a few drops of 0.2 M HCl; glutathione (10 mM) was prepared from solids (Sigma Chemical Co., St. Louis, Mo.) in extra pure water; urate (uric acid) (2 mM) and tyrosine (20 mM) were prepared from solids (BDH Chemicals Led.) in extra pure water to which a few drops of 1 M NaOH had been added to facilitate dissolving. The stock solutions of cysteine, glutathione, urate (uric acid), and tyrosine were aliquoted (separately), stored at −70° C., and thawed just before use. Ascorbate (ascorbic acid) (10 mM, from D-L extra pure crystals; Merck, Darmstadt, Germany) was prepared in extra pure water just before use.

The HPLC system consisted of a solvent pump and injector with sample cooling function (Waters Alliance), a C18 precolumn (5 μM, 3.9×20 mm; Guard-Pak Waters Sentry), and a reversed-phase C18 analytical column (5 μM, 5×250 mm; Resolve, Isco Inc.). The detector (996 PDA; Waters), UV detector, and electrochemical detector were connected in series. The output was calculated on computer (Millennium software, version 3.05.07; Waters Alliance) and the peak areas were recorded. The mobile phase was 0.2 M KH₂PO₄—H₃PO₄ (Merck, Darmstadt, Germany) at pH 2.7, delivered at 1 ml/min. The injection volume was 20 μl, and the retention times for Cysteine, ascorbate (ascorbic acid), glutathione, urate (uric acid), and tyrosine were, respectively, 3.0, 4.2, 5.0, 9.4, and 10.7 minutes, indicating good separation and clear identification of these antioxidants. The injection interval used was 12 minutes. Calibration and precision were performed on each occasion using 2.5, 5.0, 7.5, and 10.0 μl injection volumes of the freshly prepared combined standard.

The distributions of the data were not significantly different from normal (one-sample Kolmogorov-Smimov D tests, P>0.05), and parametric tests were therefore used. Results were statistically analyzed using the paired t-test to detect differences between tears collected by the two methods and differences between reflex and basal tears collected using Schirmer strips. P<0.05 was regarded as statistically significant.

Experimental Results

Precision was acceptable for all antioxidants: within-run and between-run coefficients of variation were, respectively, less than 3.0% (n=10) and 10.0% or less (n=5) in each case. No absorption peaks were obtained in three sets of blank sample, indicating no antioxidant contamination. Results showed 100% recovery of all antioxidant components in antioxidant standard from samples collected into capillary tubes or mixed with local anesthetic. Antioxidant standards onto Schirmer strips and eluted, as described earlier, showed more than 90% recovery for ascorbate (ascorbic acid), glutathione, and tyrosine and approximately 60% recovery for both cysteine and urate (uric acid). The relatively low recovery could indicate poor elution due to binding of cysteine and urate (uric acid) onto the strip. However, cysteine is unstable, and it is likely that some was degraded during collection and elution from the strip.

Table 1 presents antioxidant concentrations in human tears collected by capillary tubes and Schirmer strips. Results show that the levels of cysteine, ascorbate (ascorbic acid), glutathione, and tyrosine were significantly (P<0.01) higher in reflex tear samples collected by Schirmer strips without local anesthetic compared with tears collected concurrently by capillary tubes from the other eye of the same subject. Urate (uric acid) levels were slightly but non-significantly higher in reflex tears collected by the Schirmer strip method (P=0.1573). These results indicate release of intracellular antioxidants into the reflex tears during the period of contact between the Schirmer strip and the palpebral conjunctiva. In basal tears collected by Schirmer strips, antioxidant levels were apparently higher than in the reflex tears collected onto Schirmer strips; however, when concentrations were corrected for volume (which was lower in the case of basal tears) a basal-reflex concentration ratio of approximately 1.0 was found for each antioxidant. This indicates that the traumatic effect of the Schirmer strip was the same, whether or not anesthetic was used, and that the different molar concentrations found in basal and reflect tears collected by Schirmer strips were due to the greater dilution of released intracellular antioxidants in reflex tears.

TABLE 1

| | Reflex Tears | Reflex Tears Collected by | Basal Tears Collected |
|---|---|---|---|
| | Collected by | Schirmer Strips without | by Schirmer Strips with |
| | Capillary Tubes | Local Anesthetics | Local Anesthetic |
| | (μmol/l) | (μmol/l) | (μmol/l) |
| Cysteine | 0.7 ± 0.2 | 6.0 ± 1.7 | 12.5 ± 8.9 |
| Ascorbate | 24.4 ± 11 | 120.4 ± 108.0 | 393.3 ± 412.8 |
| Glutathione | 3.7 ± 2.5 | 25.1 ± 16.8 | 75.8 ± 66.8 |
| Urate | 98.1 ± 41.9 | 116.5 ± 74.2 | 166.0 ± 83.5 |
| Tyrosine | 2.7 ± 1.3 | 10.8 ± 4.8 | 29.8 ± 18.7 |

II. Antioxidant Protection and UV Absorption: A Comparison Between Normal Human Tears and Commercial Artificial Tears
Normal Human Tear Collections

A total of 120 Chinese subjects were recruited with their informed consent. There were 58 young subjects (32 males, 26 females; mean±SD age 23±3 years) and 62 older subjects (12 males, 50 females; mean±SD age 58±6 years). None of the subjects were contact lens wearers, and all reported good general health. Each subject was required to have a preliminary eye examination at the Optometry Clinic of The Hong Kong Polytechnic University to ensure normal ocular health prior to entry into the study. A simple questionnaire regarding experience of dry eye symptoms was also administered.

For each sampling, a disposable capillary tube (Drummond Scientific Co. USA) was gently placed on the outer canthus of one eye, randomly selected, of each subject to simulate itching sensation for tear stimulation, and about 30 μl of reflex tears was collected from the other, non-stimulated eye using 10 μl disposable capillary tubes (Drummond Scientific Co. USA) (see Callender and Morrison 1974). The time to collect 30 μl of tears was recorded to monitor the tear flow rate.

Commercial Artificial Tear Preparations

Artificial tears from 14 commercially available ophthalmic preparations marketed for alleviation of dry eye symptoms were tested. Table 2 lists these commercial artificial tear preparations and their active ingredients (source of information from manufacturers' brochures or instruction leaflets).

TABLE 2

| | Active agent (as stated by manufacturer in |
|---|---|
| Preparation | package insert or on pack) |
| Bion tears ® | Dextran, Hydroxypropyl Methycellulose 2910, electrolytes |
| Blink ™ eye drops | Polyvinyl alcohol, Chlorite/peroxy compound, electrolytes |
| Cool eye | Hydroxypropyl Methylcellulose, Benzalkonium Chloride |
| Duratears ® | White Petrolatum, Anhydrous Liquid Lanolin, Mineral Oil |
| Genteal ™ lubricant eye drop | Hydroxypropyl Methylcellulose, Boric acid, electrolytes |
| Hypotears ® Plus | Polyvidone, electrolytes |
| Isopto ® tears | Hydroxypropyl Methylcellulose, Benzalkonium Chloride |
| Lacryvisc ® opthalmic gel | Carbomer 974P, Sorbitol, sodium hydroxide |
| Opti-tears ® | Dextran, Hydroxypropyl Methycellulose 2910, Edetate |
| | disodium, Polyquad |
| Protagent ® Eye Drops | Povidone, Benzalkonium Chloride |
| Tear Naturale ™ Free | Dextran, Hydroxypropyl Methycellulose 2910 |
| Tear Naturale ® II | Dextran 70, Hydroxypropyl Methycellulose 2910, Polyquad |
| Viscotears ® | Polyacrylic acid, carbomer, Cetrimide |
| VisMed ® | Sodium hyaluronate |

Total Antioxidant Status Measurement

The artificial tear preparations, together with the normal human tear samples, were analyzed for total antioxidant capacity as the Ferric Reducing/Antioxidant Power (FRAP) value using the FRAP assay (see Benzie and Strain, 1999; Choy et al., 2000).

UV Absorbance Measurement

To investigate the ability of the artificial tears to prevent photo-oxidation, UV/low visible absorbance spectrum (230-400 nm) of each artificial tear preparation was assessed, using a Hewlett Packard 8543 UV-visible spectrophotometric system (Hewlett Packard, USA), and results were compared with the UV absorbance of pooled human tears (from 10 healthy volunteers).

Statistics

The distribution of the data was not significantly different from normal (One Sample Kolmogorov-Smirnov D Tests, P>0.05) and thus, parametric tests were used. Unpaired t-tests were used to investigate differences between the two age groups and male-female differences. Chi-square test was used in a sub-analysis of age and tear flow rate. A value of P<0.05 was regarded as significant.

Experimental Results

In-run and between-run coefficients of variation for the FRAP assay were both less than 3% (n=10). The limit of detection was 2.5 μM (determined by mean±3 SD of the signal given by pure distilled water, n=10). For the tear analysis, subjects showed high within- and between-group variations of tear flow rate.

1. Impact of Age on Normal Human Tears: Flow Rate and Antioxidant Status

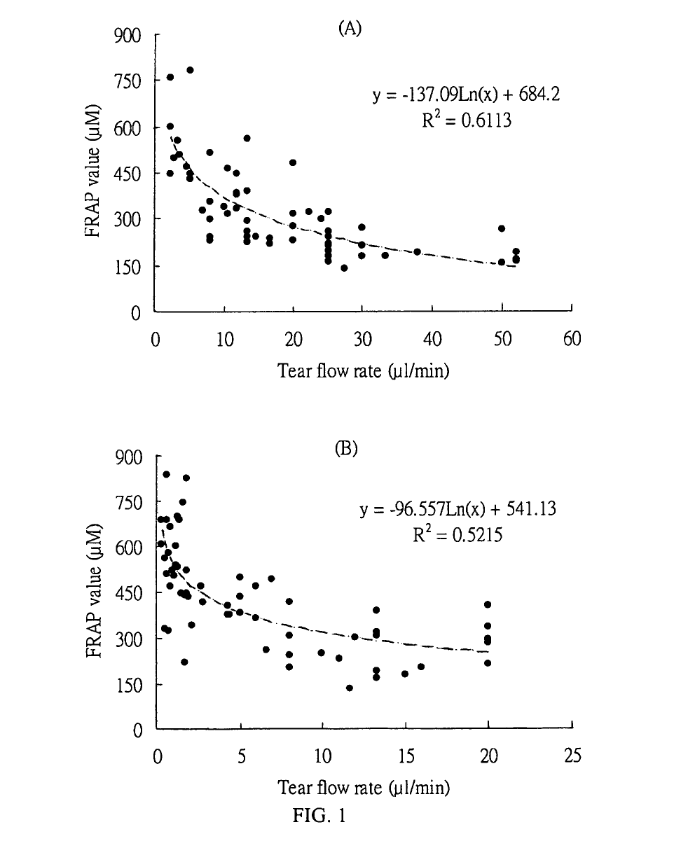

Applicants' unpublished data showed an age-related decrease in both flow rate and total antioxidant status of human tears. No gender difference was observed in either variable. As illustrated in FIG. 1, tear total antioxidant capacity as the FRAP values varied widely between individuals, ranging from 133 to 835 μM, and were significantly inversely correlated with tear flow rates in both the younger age (r=−0.782; P<0.0001) and the older age (r=−0.722; P<0.0001) groups. Tear flow rates were also highly variable, but were significantly (P<0.0001) lower in older subjects. Almost half (43%) of the older subjects, but none of the younger subjects, had tear flow rate of <2 μl/min. Conversely, none of the older subjects, but 36% of the younger subjects had tear flow rate >20 μl/min.

A sub-analysis of tear antioxidant status was performed based on tear flow rate. Table 3 shows that the total antioxidant status in the tears of older subjects was significant lower than that in the tears of younger subjects with similar tears flow rate (P<0.05).

When male-female differences were explored, no significant differences were found in total antioxidant status (P>0.05). The tear total antioxidant status in the tears of older women was significantly lower than that in younger women with similar tear flow rates (P=0.017), however, the difference in the tears of young versus older men did not reach statistical significance. None of the subjects in the younger age group reported experiencing dry eye symptoms. In the older age group, no men reported symptoms, but 13 of the women reported experiencing symptoms of dry eye. In women with dry eye symptoms, the tear total antioxidant capacity as the FRAP value was slightly but not significantly lower than those who did not experience symptoms (P=0.0774); Mean±SD values were, respectively, 350±115 μM (n=13) and 446±180 μM (n=37). Interestingly, the flow rate in the women with dry eye symptoms (9.7±6.1 μl/min) was not lower than that of the women without symptoms (6.1±6.9 μl/min) (P=0.1068).

TABLE 3

| | | | | Flow Rate | FRAP value |
|---|---|---|---|---|---|
| | | | Subject | Mean ± SD (range) | Mean ± SD |
| | | | number | μl/min | μM |
| High tear | Younger | Overall | n = 21 | 33.3 ± 10.8 (24.0-52.0) | 207 ± 49 |
| flow rate | | Male | n = 8 | 33.2 ± 11.4 (25.0-52.0) | 216 ± 52 |
| (>25 μl/min) | | Female | n = 13 | 33.3 ± 10.9 (24.0-52.0) | 202 ± 48 |
| | Older | — | None | — | — |
| Medium | Younger | Overall | n = 37 | 10.6 ± 5.7 (2.3-22.2) | 387 ± 143* |
| tear flow | | Male | n = 24 | 11.6 ± 5.7 (2.3-22.2) | 368 ± 138 |
| rate | | Female | n = 13 | 8.7 ± 5.5 (2.3-20) | 423 ± 150# |
| (2-24 μl/min) | Older | Overall | n = 35 | 10.7 ± 6.0 (2.1-20.0) | 321 ± 98 |
| | | Male | n = 5 | 9.1 ± 4.8 (4.4-15) | 276 ± 119 |
| | | Female | n = 30 | 11.0 ± 6.2 (2.1-20) | 329 ± 95 |
| Low tear | Younger | — | None | — | — |
| flow rate | Older | Overall | n = 27 | 1.1 ± 0.5 (0.3-1.9) | 552 ± 146 |
| (<2 μl/min) | | Male | n = 7 | 1.1 ± 0.7 (0.3-1.8) | 527 ± 82 |
| | | Female | n = 20 | 1.1 ± 0.5 (0.3-1.9) | 561 ± 164 |
| | | Female without dry | n = 37 | 6.1 ± 6.9 (0.3-20) | 447 ± 180 |
| | | eye symptoms | | | |
| | | Female with dry | n = 13 | 9.7 ± 6.1 (1.5-20) | 350 ± 115 |
| | | eye symptoms | | | |

*At similar flow rates, the FRAP value of the younger age group was significantly higher than that of the elderly group (P = 0.0264).
#At similar flow rates, the FRAP value of the young females was significantly higher than that of the old females (P = 0.0170).

An age-related decrease in tear antioxidant status shown in our results (not shown) suggests that the ocular surfaces of elderly individuals are under higher risk of oxidative damage. The data disclosed here are consistent with previous findings in ocular tissues or fluids which show that the levels of some antioxidants in the aqueous and crystalline lens decrease with age (Bates and Cowen, 1988; Zigman and Schultz, 1998).

Acorbate (ascorbic acid) is believed to be the component that accounts for an age-related decrease in tear antioxidant status. Ascorbate (Vitamin C; ascorbic acid) is important in suppressing inflammatory response and in enhancing wound healing in the cornea (Kasetsuwan et al. 1999). Furthermore, multi-vitamin supplementation has been shown to improve tear quality in terms of tear stability (Patel et al. 1993). In the present invention, inclusion of Vitamin C as antioxidant in artificial tears is considered a preferred embodiment for the purpose of increasing the effectiveness of artificial tears for the treatment of dry eye.

Another preferred antioxidant for artificial tears according to the present invention is lactoferrin, an iron-binding antioxidant. Tear lactoferrin has been reported to be lower in elderly subjects and in patients suffering from dry eye (Ohashi 2003).

2. Commercial Artificial Tears: Total Antioxidant Status and UV Absorbance Characteristics

Among the 14 commercially available artificial tear preparations tested, none provides the same kind of anti-oxidative protection as afforded by Mother Nature to human tears. Apparently, antioxidant capacity has not been given due consideration in formulating artificial tears.

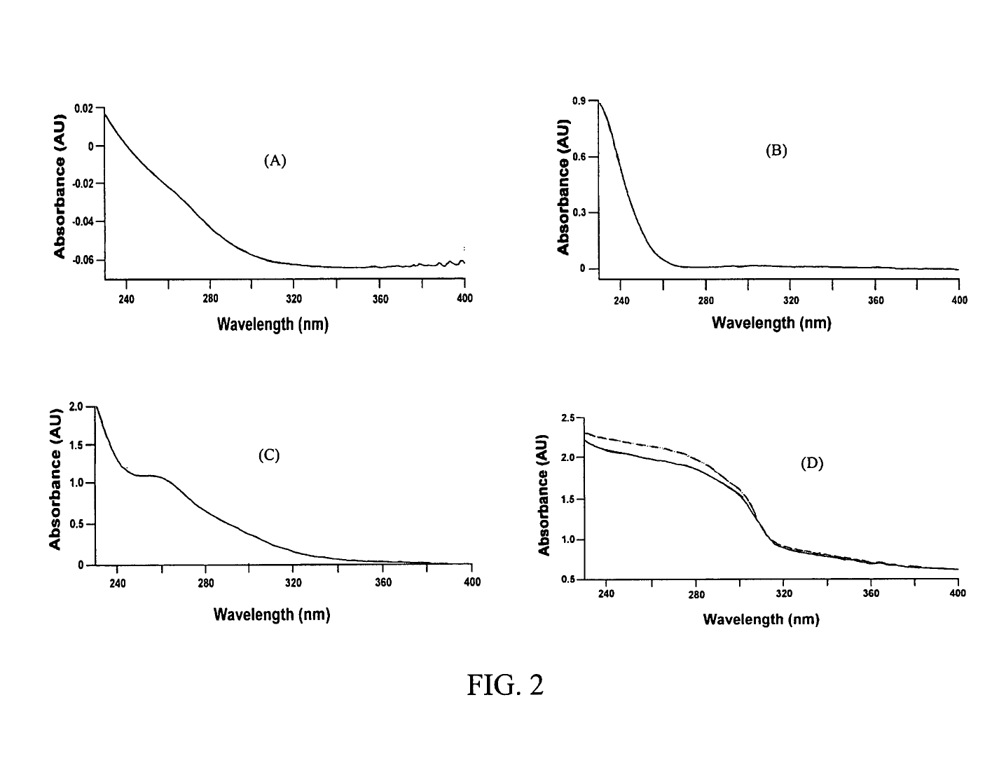

With respect to UV absorption ability, human tears were found to absorb light of 230-400 nm, while the absorption spectra of most existing artificial tears tested were quite different (FIG. 2).

Solar ultraviolet (UV) radiation can initiate the processes of photo-oxidation and is thus a powerful ROS generator (Halliwell and Gutteridege 1999). Because the eye is the unique organ that directly lets light pass through from cornea to retina, attenuation of UV light entering the eye is important.

Inventors' unpublished experimental results show that most commercially available artificial tear preparations tested have no detectable antioxidant capacity and have a UV absorption profile that is quite different from that of fresh human tears. This indicates that the use of currently available commercials artificial tears will compromise both the antioxidant protection to the anterior surface of the eye and the first layer of UV absorption, due to dilution of the tear film by liquid containing no significant antioxidant capacity and little or no UV absorption power.

III. “Natural Balance” Artificial Tear Formulation, Preparation, and Administration for Human Subjects

The above experimental data show that elderly people have lower tear flow rate and that their tear have lower anti-oxidative protection, and that most of commercially available, artificial tears lack both the antioxidant capacity and UV absorbing characteristics of natural tears. On application into the eye, they will reduce the protection offered by natural tears. The present invention provides a ‘natural balance’ artificial tear formulation that helps to restore antioxidant and UV absorbing properties to the tear film of the aging eye, thereby helping prevent or improve dry eye symptoms in the elderly, as well as and promote ocular health in general regardless of age.

As a preferred embodiment, the ophthalmic composition of the present invention is similar to natural tears in terms of anti-oxidative protection and UV filtering profiles, providing a ‘natural balance’ artificial tear fluid that will be beneficial to the millions of regular artificial tear users worldwide. As one embodiment, the ophthalmic composition comprises a physiologically balanced salt solution and one or more anti-oxidative ingredients which collectively confer said ophthalmic composition a total antioxidant capacity as the FRAP value greater than 100 μmol/l (preferably, greater than 150 μmol/l), and at least one ingredient having peak UV absorption in the range of 200-350 nm.

The anti-oxidative ingredient of the ophthalmic composition, according to the invention, includes, but is not limited to, ascorbate (ascorbic acid), urate (uric acid), cysteine, glutathione, tyrosine, lactoferrin, transferrin, albumin, caeruloplasmin, and lacrimal gland peroxidase. Ascorbate (ascorbic acid), urate (uric acid), cysteine, glutathione, and tyrosine also function as UV filters, having range of UV absorption from 230 to 320 nm.

The term “UV filter” used here means a substance that absorbs UV light in the range between 200 and 350. Thus, when present in the artificial tear formulation, a UV filter reduces the harmful ultraviolet light entering the eyes.

“Physiologically balanced salt solution” here means that osmolarity, pH and electrolyte concentrations are similar to those of normal human tears. A physiologically balanced salt solution serves as a base carrier for active ingredients of artificial tears and may comprise one or more of the following components: electrolyte (or salt), water, methylcellulose, polyvidone, carbomer, mineral oil, etc. People with ordinary skill in the art are able to prepare a physiologically balance salt solution for ophthalmic use.

The physiologically balanced salt solution of the ophthalmic composition according to the invention may contain various additives as needed, which include buffering agents, isotonizers, preservatives, thickeners, stabilizers, pH-adjusting agents, chelating agents, etc.

Buffering agents are added to keep the pH within a narrow range, and include carbonate buffer, borate buffer, citrate buffer, tartrate buffer, phosphate buffer, acetate buffer, and others. Such a buffer is added in an amount that is suitable for the desirable pH range to be maintained.

Isotonizers are added to make the preparation isotonic with the tears, such as, polyethylene glycol, propylene glycol, etc.; and electrolytes such as sodium ion, chloride ion, potassium ion, etc. Such an isotonizer is added in an amount that makes the osmotic pressure of the eye drop equal to that of the tear. Preservatives that may be used are benzalkonium chloride, parabens, chlorobutanol, polyqual, edetate disodium, etc.

Thickeners or viscosity-increasing components may be added to increase viscosity, including a cellulose ester (e.g., hydroxypropyl methylcellulose, hydroxy cellulose, methyl cellulose, carboxymethyl cellulose, and hydroxyethyl cellulose carboxymethyl cellulose), polyvinyl alcohol, providone, polyacrylic acid, hyalurnoic acid, and chondroitin sulfate, glycerol, carboxyvinyl polymers, etc. Preferably, the composition has a viscosity in the range of 10-25 centipoise.

Other additives that may be added, if desired, include: a lipid component (e.g., castor oil, mineral oil, olive oil); stabilizers such as sodium sulfite, propylene glycol, etc.; pH-adjusting agents such as hydrochloric acid, citric acid, phosphoric acid, acetic acid, tartaric acid, sodium hydroxide, potassium hydroxide, sodium carbonate, sodium bicarbonate, etc.; and chelating agents such as sodium edetate, sodium citrate, etc.

The ophthalmic composition according to the invention is prepared by aseptic manipulation, or sterilization is performed at a suitable stage of preparation.

A preferred example of the ophthalmic formulation for use as artificial tears of the present invention comprising the following ingredients:
ascorbate (ascorbic acid): 25 μmol/l;
urate (uric acid): 100 μmol/l;
cysteine: 0.7 μmol/l;
glutathione: 4.0 μmol/l;
tyrosine: 3.0 μmol/l;
lactoferrin: 1645 μg/ml;
transferrin: 15.5 μg/ml;
albumin: 40 μg/ml;
caeruloplasmin: 210 μg/ml;
lacrimal gland peroxidase: 7.0 μg/ml, and
a physiologically balanced salt solution, which may include one or more of the following substances: electrolyte, water, methylcellulose, polyvidone, Carbomer, Mineral oil, etc., prepared in a way so that the solution is suitable as a carrier for the above listed ingredients for ophthalmic applications. For other ophthalmic solutions, the exact quantities might vary.

Some antioxidants, such as ascorbate (ascorbic acid), are very unstable and can degrade rapidly unless the solution is highly acidic. However, it is undesirable for artificial tears to be highly acidic. To solve this problem, a storage and delivery vessel for a two-part medicament, as disclosed in the U.S. patent application Ser. No. 11/142,396, can be used. Said vessel comprises a first deformable chamber for holding a first part of a medicament, a dispensing aperture, and a closure separating the first and second chambers and adapted to open or rupture when the first chamber is deformed by an external force so as to mix the first and second parts of the medicament for dispensing from the dispensing aperture. In applying the vessel to the present invention, the active ingredient, such as ascorbate (ascorbic acid), and the salt solution are stored in separate chambers. Therefore, the active ingredient is stored in a relative dry condition under which it can be stable for a long time and is mixed with the salt solution only for a short period (for example, less than a day) before being applied to human eyes.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims.

### REFERENCES

1. Kirchner R. Editorial. News Review. Rev Optom 1997;October: 7.
2. Lin P Y, Tsai S Y, Cheng C Y, Liu J H, Chou P, Hsu W M Prevalence of dry eye among an elderly Chinese population in Taiwan: The Shihpai eye study. Ophthalmology 2003;110:1096-101.
3. Schein O D, Munoz B, Tielsch J M, Bandeen-Roche K, West S. Prevalence of dry eye among the elderly. Am J Ophthalmol 1997;124:723-8.
4. Lemp M A. Report of the National Eye Institute/Industry Workshop on clinical trials in dry eyes. Clao J 1995;21:221-32.
5. Goto E, Yagi Y, Matsumoto Y, Tsubota K. Impaired functional visual acuity of dry eye patients. Am J Ophthalmol 2002;133:181-6.
6. Lemp M A. Recent developments in dry eye management. Ophthalmology 1987; 94:1299-304.
7. Tsubota K, Satake Y, Shimazaki J. Treatment of severe dry eye. Lancet 1996; 348:123.
8. Albietz J M. Dry eye: an update on clinical diagnosis, management and promising new treatments. Clin Exp Optom 2001;84:4-18.
9. Ohashi Y, Ishida R, Kojima T, Goto E, Matsumoto Y, Watanabe K, et al. Abnormal protein profiles in tears with dry eye syndrome. Am J Ophthalmol 2003;136:291-9.
10. Augustin A J, Spitznas M, Kaviani N, Meller D, Koch F H, Grus F, et al. Oxidative reactions in the tear fluid of patients suffering from dry eyes. Graefe's Arch Clin Exp Ophthalmol 1995;233:694-8.
11. Rieger G. Anti-oxidative capacity of various artificial tear preparations. Graefes Arch Clin Exp Ophthalmol 2001;239:222-6.
12. Benzie I F F, Strain J J. Ferric reducing/antioxidant power assay: direct measure of total antioxidant activity of biological fluids and modified version for simultaneous measurement of total antioxidant power and ascorbic acid concentration. Methods Enzymol 1999;299:15-27.
13. Choy C K M, Benzie I F F, Cho P. Ascorbic acid concentration and total antioxidant activity of human tear fluid measured using the FRASC assay. Invest Ophthalmol Vis Sci 2000;41:3293-8.
14. Bates C J, Cowen T D. Effects of age and dietary vitamin C on the contents of ascorbic acid and acid-soluble thiol in lens and aqueous humour of guinea-pigs. Exp Eye Res 1988;46:937-45.
15. Zigman S, Schultz J B, Schultz M. Measurement of oxygen production by in vitro human and animal lenses with an oxygen electrode. Curr Eye Res 1998;17:115-9.
16. Choy C K M, Cho P, Benzie I F F. Water-soluble antioxidants in human tears: effect of collection method. Invest Ophthalmol Vis Sci 2001;42:3130-4.
17. Gogia R, Richer S P, Rose R C. Tear fluid content of electrochemically active components including water soluble antioxidants. Curr Eye Res. 1998; 17:257-263.
18. Kuzuya M, Ando F, Iguchi A, Shimokata H. Effect of aging on serum uric acid levels: longitudinal changes in a large Japanese population group. J Gerontol A Biol Sci Med Sci 2002;57:M660-4.
19. Kasetsuwan N, Wu F M, Hsieh F, Sanchez D, McDonnell P J. Effect of topical ascorbic acid on free radical tissue damage and inflammatory cell influx in the cornea after excimer laser corneal surgery. Arch Ophthalmol 1999;117:649-52.
20. Patel S, Plaskow J, Ferrier C. The influence of vitamins and trace element supplements on the stability of the pre-corneal tear film. Acta Ophthalmol 1993;71:825-9.
21. Halliwell B, Gutteridge J M C. Free Radicals in Biology and Medicine. 3rd ed. Oxford: Oxford University Press; 1999.
22. Kirshenbaum A, Donald M. Atlas of protein spectra in the ultraviolet and visible regions. New York: IFI/Plenum; 1994.
23. Ringvold A, Anderssen E, Kjonniksen I. UV Absorption by Uric Acid in Diurnal Bird Aqueous Humor. Invest Ophthalmol Vis Sci 2000;41:2067-9.

What is claimed is:

1. An ophthalmic composition comprising a physiologically balanced salt solution and one or more anti-oxidative ingredients which collectively confer said ophthalmic composition a total antioxidant capacity expressed as a FRAP value of greater than 100 μmol/l.

2. The ophthalmic composition of claim 1, wherein at least one ingredient has peak UV absorption properties in the range of 200-350 nm.

3. The ophthalmic composition of claim 2, wherein at least one anti-oxidative ingredient is selected from the group consisting of ascorbate (ascorbic acid), urate (uric acid), cysteine, glutathione and tyrosine.

4. An ophthalmic composition, which comprises a physiologically balanced salt solution and at least a first anti-oxidative ingredient selected from the group consisting of:
ascorbate (ascorbic acid): 10-80 μmol/l;
urate (uric acid): 50-200 μmol/l;
cysteine: 0.4-1.0 μmol/l;
glutathione: 1.5-11.0 μmol/l; and
tyrosine: 1.0-6.0 μmol/l.

5. The ophthalmic composition of claim 4, further comprising a second anti-oxidative ingredient selected from the group consisting of:
ascorbate (ascorbic acid): 10-80 μmol/l;
urate (uric acid): 50-200 μmol/l;
cysteine: 0.4-1.0 μmol/l;
glutathione: 1.5-11.0 μmol/l; and
tyrosine: 1.0-6.0 μmol/l.

6. The ophthalmic composition of claim 5, further comprising a third anti-oxidative ingredient selected from the group consisting of:
ascorbate (ascorbic acid): 10-80 μmol/l;
urate (uric acid): 50-200 μmol/l;
cysteine: 0.4-1.0 μmol/l;
glutathione: 1.5-11.0 μmol/l; and
tyrosine: 1.0-6.0 μmol/l.

7. The ophthalmic composition of claim 6, further comprising a fourth anti-oxidative ingredient selected from the group consisting of:
ascorbate (ascorbic acid): 10-80 μmol/l;
urate (uric acid): 50-200 μmol/l;
cysteine: 0.4-1.0 μmol/l;
glutathione: 1.5-11.0 μmol/l; and
tyrosine: 1.0-6.0 μmol/l.

8. The ophthalmic composition of claim 4, comprising the following five anti-oxidative ingredients:
ascorbate (ascorbic acid): 10-80 μmol/l;
urate (uric acid): 50-200 μmol/l;
cysteine: 0.4-1.0 μmol/l;
glutathione: 1.5-11.0 μmol/l; and
tyrosine: 1.0-6.0 μmol/l.

9. The ophthalmic composition of claim 8, wherein ascorbate (ascorbic acid) is 25 μmol/l; urate (uric acid) is 100 μmol/l; cysteine is 0.7 μmol/l; glutathione is 4.0 μmol/l; and tyrosine is 3.0 μmol/l.

10. The ophthalmic composition of claim 4, further comprising one or more ingredients selected from the group consisting of:
lactoferrin: 1500-1790 μg/ml;
transferrin: 13-18 μg/ml;
albumin: 37-47 μg/ml;
caeruloplasmin: 8-14 μg/ml; and
lacrimal gland peroxidase: 4-7 μg/ml.

11. The ophthalmic composition of claim 10, wherein lactoferrin is 1600-1700 μg/ml; transferring is 15-16 μg/ml; albumin is 40-45 μg/ml; caeruloplasmin is 200-220 μg/ml; and lacrimal gland peroxidase is 5-7 μg/ml.

12. The ophthalmic composition of claim 1, further comprising a viscosity-increasing component.

13. The ophthalmic composition of claim 12, wherein the viscosity-increasing component is selected from the group consisting of a cellulose ester, polyvinyl alcohol, providone, polyacrylic acid, hyalurnoic acid, and chondroitin sulfate.

14. The ophthalmic composition of claim 13, wherein the cellulose ester is selected from the group consisting of hydroxypropyl methylcellulose, hydroxy cellulose, methyl cellulose, carboxymethyl cellulose, and hydroxyethyl cellulose.

15. A method of treating dry eye symptoms or improving ocular health in a human eye comprising a step of administering to said eye an ophthalmic composition comprising a physiologically balanced salt solution and one or more anti-oxidative ingredients which collectively confer said ophthalmic composition a total antioxidant capacity expressed as a FRAP value greater than 100 μmol/l.

16. The method of claim 15, wherein at least one anti-oxidative ingredient is selected the group consisting of ascorbate (ascorbic acid), urate (uric acid), cysteine, glutathione and tyrosine.

17. The method of claim 16, wherein one anti-oxidative ingredient is ascorbate (ascorbic acid) with a concentration between 10-80 μmol/l.

18. The method of claim 15, further comprising a step of mixing said a physiologically balanced salt solution and said one or more anti-oxidative ingredients within one day before said step of administering to said eye.

19. The method of claim 1, wherein said FRAP value is greater than 150 μmol/l.

20. The method of claim 15, wherein said FRAP value is greater than 150 μmol/l.
